(19) United States
(12) Patent Application Publication
(10) Pub. No.: US 2022/0162382 A1
(43) Pub. Date: May 26, 2022

(54) PRODUCTION OF NANOPARTICLES AND MICROPARTICLES

(71) Applicant: UCL BUSINESS LTD, London (GB)

(72) Inventors: Giuseppe BATTAGLIA, London (GB); Aroa Duro CASTANO, London (GB)

(73) Assignee: UCL BUSINESS LTD, London (GB)

(21) Appl. No.: 17/608,247

(22) PCT Filed: May 1, 2020
(86) PCT No.: PCT/GB2020/051077

§ 371 Date: Nov. 2, 2021

(30) Foreign Application Priority Data
May 3, 2019 (GB) 1906299.1

(51) Int. Cl. C08G 69/10; C07K 14/00; C08G 65/334

(52) U.S. Cl. CPC C08G 69/10; B82Y 40/00; C08G 65/3346; C07K 14/001

(57) ABSTRACT

The present invention is directed to a method for producing nanoparticles and microparticles composed of peptide- or peptoid-containing amphiphilic polymers. The method is simple, capable of achieving high yields, and can be tailored to produce a range of industrially and therapeutically useful structures including vesicles, micelles and hydrogels. The present invention also provides related hydrogels and vesicles having beneficial properties such an ability to degrade and release a payload in response to external stimuli.

### FIELD OF THE INVENTION

The present invention is directed to a method for producing nanoparticles and microparticles composed of peptide- or peptoid-containing amphiphilic polymers. The present invention also provides related hydrogels and vesicles having beneficial properties such an ability to degrade and release a payload in response to external stimuli.

### BACKGROUND OF THE INVENTION

Ring Opening Polymerization (ROP) of amino acid N-carboxyanhydrides (NCAs) is a widely applied technique to produce polypeptides. Polypeptides produced from NCAs can form secondary structures, bear multiple functionalities, have adjustable molecular weights (e.g., 1-1000 kDa) and structural homogeneity that favour self-assembly into defined nano structures.

Synthesis of narrowly distributed polypeptides is a desirable goal, but has turned out to be rather demanding due to the co-existence of several reaction mechanisms and a large number of possible side reactions. There are many factors hindering the controlled polymerization such as purity of NCAs, reaction system (purity of solvents, initiator chosen, presence of moisture, CO2 pressure, pH, temperature), presence of salts, cleavage of protecting groups, and undesired termination processes (carbamate mechanisms or reaction with α-isocyanatocarboxylates).

Separately, self-assembled amphiphilic block copolymers, in general, have been created using a range of different hydrophilic and hydrophobic polymer blocks. Such compounds can be used to generate nanoparticle and microparticle structures that have a range of utilities, including but by no means limited to the creation of hydrogels and the creation of therapeutic vesicles that may harbour encapsulated therapeutic agents or even be therapeutic in their own right (for instance, via the release of pharmaceutically active agents as they degrade in vivo). Until now, self-assembled structures of this type have often been produced first by synthesising their constituent amphiphilic polymers, and then by converting the polymers into self-assembled structures post-polymerization (e.g., by film rehydration methods). However, self-assembly of amphiphilic block copolymers in this way is usually limited to dilute copolymer solutions (<1%), which represents a significant disadvantage for potential commercial applications due to obvious problems in manufacture and scaling up processes.

Meanwhile, polymerization-induced self-assembly (where self-assembly occurs as the polymerization is ongoing) is known in the bulk for certain toughened thermosets and elastomers formed by step polymerization. Nevertheless, it has never been applied to the synthesis of polypeptide-based nanoparticles such as those made by ring opening polymerization techniques.

There is consequently a need for improved methods for producing nanoparticles and microparticles based on peptide-containing amphiphilic polymers. It would also be desirable to provide peptide-containing nanoparticles and microparticles that have beneficial properties.

### SUMMARY OF THE INVENTION

The present invention addresses these problems via the provision of a synthetic method for producing nanoparticles and microparticles composed of peptide- or peptoid-containing amphiphilic polymers. The method is simple, capable of achieving high yields, and can be tailored to produce a range of industrially and therapeutically useful structures including vesicles, micelles and hydrogels. The present invention also provides related hydrogels and vesicles having beneficial properties such an ability to degrade and release a payload in response to external stimuli.

Specifically, the present invention provides, in a first aspect, a method of preparing self-assembled nanoparticles or microparticles, wherein:
said self-assembled nanoparticles or microparticles comprise amphiphilic copolymers each comprising a hydrophilic polymer block and a hydrophobic polymer block;
said hydrophobic polymer block comprises a polypeptide or polypeptoid; and
said method comprises:
(i) providing, in a polar aprotic solvent, hydrophilic polymer blocks as initiator molecules;
(ii) contacting said hydrophilic polymer blocks with hydrophobic polymer block precursor monomers, and forming said hydrophobic polymer blocks by polymerization reactions, initiated at the hydrophilic polymer blocks, of said hydrophobic polymer block precursor monomers, thereby producing said amphiphilic copolymers; and
(iii) allowing said amphiphilic copolymers to self-assemble in situ to form said self-assembled nanoparticles or microparticles.

Preferably the method further comprises transferring (e.g., by membrane dialysis, ultrafiltration, size exclusion chromatography or tangential flow filtration) said self-assembled nanoparticles or microparticles into an aqueous medium by displacement of the polar aprotic solvent.

In the method of the invention, the hydrophobic polymer block precursor monomers are typically cyclic and the polymerization reactions are ring-opening polymerization (ROP) reactions. For instance, the hydrophobic polymer block precursor monomers may be amino acid N-carboxyanhydrides.

In certain preferred aspects of the method of the invention, the hydrophobic polymer block comprises a polypeptide. For instance, in exemplary aspects the polypeptide comprises amino acid residues selected from the group consisting of methionine, histidine, lysine, glutamic acid, phenylalanine and derivatives thereof. Particularly preferred polypeptides include: (a) a polypeptide that comprises methionine; (b) a polypeptide that comprises pendant groups having a pKa in the range 4.0 to 7.5; and/or (c) a peptide that comprises chemically reactive pendant groups suitable for further functionalising the self-assembled nanoparticles or microparticles.

In certain preferred aspects of the method of the invention, the hydrophilic polymer block comprises a polymer selected from the group consisting of polyesters, polyamides, polyanhydrides, polyurethanes, polyethers, polyimines, polypeptides, polypeptoids, polyureas, polyacetals and polysaccharides.

In one embodiment of the method of the invention, the polar aprotic solvent is selected from the group consisting of dimethyl sulfoxide, tetrahydrofuran, dioxane, N,N-dimethyl formamide, N,N-dimethyl acetamide and 1,3-dimethyl-2-imidazolidinone.

Optionally, the method comprises providing said hydrophilic polymer blocks in situ by polymerizing hydrophilic polymer block precursor monomers.

In a particularly preferred aspect of the method of the invention, the steps (i) to (iii) are carried out as a one-pot reaction.

In the method of the invention, and as explained in more detail herein, the variables such as the concentrations of the reagents and the duration of the reaction can be varied in order to control the structure of the resulting self-assembled product. For instance, the concentrations of the reagents can be adjusted to affect the solids content of the product mixture, i.e., the relative amount of the self-assembled nanoparticles or microparticles within respect to the total weight of the reaction medium. In certain aspects of the invention, the reaction steps are terminated when the self-assembled nanoparticles or microparticles constitute from 0.1 to 90% by weight, and preferably from 5 to 70% by weight, of the total weight of the reaction medium. Alternatively or additionally the degree of polymerisation of the hydrophobic polymer block is controlled to be from 5 to 400 (e.g., by controlling the duration of the reaction) and/or the degree of polymerisation of the hydrophilic polymer block is from 5 to 200.

Thus, in embodiments of the method, the resulting self-assembled nanoparticles or microparticles may comprise micelles, optionally wherein the micelles collectively form a gel. Alternatively, the resulting self-assembled nanoparticles or microparticles may comprise vesicles.

In another aspect, the present invention provides a hydrogel comprising a plurality of micellar nanoparticles or microparticles, wherein said nanoparticles or microparticles each comprise amphiphilic copolymers each comprising a hydrophilic polymer block and a hydrophobic polymer block that comprises a polypeptide or polypeptoid. Such hydrogels can be prepared by carrying out the method of the invention under appropriate reaction conditions, as discussed in more detail later herein.

In a still further aspect, the present invention provides a vesicle that comprises an amphiphilic block copolymer comprising a hydrophilic block and a hydrophobic block that comprises a polypeptide or polypeptoid, wherein said vesicle is suitable for administration to a subject and said hydrophobic block is capable of undergoing a chemical transformation, leading to degradation of said vesicle in vivo, in response to a change in in vivo conditions.

In one exemplary such vesicle, the change in in vivo conditions is a change in pH and the hydrophobic block comprises pendant groups having a pKa in the range 4.0 to 7.5. For instance, the pendant groups optionally comprise imidazolyl groups, preferably wherein at least some of said imidazolyl groups are provided by histidine residues in said hydrophobic block.

In another exemplary such vesicle, the change in in vivo conditions is an increase in the concentration of reactive oxygen species (ROS). For instance, the hydrophobic block may comprise methionine residues.

In the vesicle of the present invention, a drug (and/or other substance including but not limited to imaging agents, probes, and targeting moieties) may be encapsulated within the vesicle or attached to the surface of the vesicle.

Preferred aspects of the present invention are further discussed below.

### BRIEF DESCRIPTION OF THE FIGURES

FIG. 1 shows a schematic representation of the “one-pot” synthesis of polypeptide-based particles by the method of the invention. A hydrophilic block is either provided as a macroinitiator or synthesised in situ from hydrophilic monomers M₁. Monomers M₂ are then polymerized (e.g., by ring opening polymerization, ROP) on the hydrophilic block to form an attached hydrophobic polymer block, and polymerisation-induced self-assembly (PISA) gives rise to self-assembled particles such as spheres/micelles (hydrophobic blocks internally, surrounded by external hydrophilic blocks), worms (“elongated” spheres/micelles; hydrophobic blocks internally, surrounded by external hydrophilic blocks) or vesicles (double layer of amphiphilic polymers; hydrophobic blocks surrounded internally and externally by hydrophilic blocks). As shown schematically in the Figure, generation of desired particles structures can be achieved by adjusting the degree of polymerization of the hydrophobic block (e.g. a polymethionine block) and total solids concentration of polymerization components compared to solvent.

FIG. 2 shows ¹H-NMR spectra of a sub-set of the amphiphilic polymers produced in Example 1, as follows: Panel (A)—polysarcosine-polymethionine; Panel (B)—poly(ethylene glycol)-polymethionine; Panel (C)—polysarcosine-polyphenylalanine; Panel (D) poly(ethylene glycol)-polyphenylalanine in TFA-d.

FIG. 3 shows ¹H-NMR spectra of poly(ethylene glycol)-polymethionine polymerization and self-assembly as per Example 2 at different time points of the reaction in TFA-d (from bottom trace to top trace being 1 hour, 2 hours 4 hours and 24 hours, respectively).

FIG. 4 shows ¹H-NMR spectra in TFA-d (A) and GPC traces (Light Scattering 90° C., in acidic water) of particular example of poly(ethylene glycol)-polymethionine of different hydrophobic block length obtained using the present method at 15% w solid content, and as per Example 2.

FIG. 5 shows particle size obtained by dynamic light scattering of polymerization induced self-assembly of poly(ethylene glycol)-polymethionine as per Example 2 at different time points in DMSO. Panel (A) shows sie distribution by number. Panel (B) shows size distribution by intensity. Panel (C) shows correlation coefficients of the measurements indicating their suitability.

FIG. 6 shows particle size obtained by dynamic light scattering of polymerization induced self-assembly of poly(ethylene glycol)-polymethionine as per Example 2 at different time points after transfer to water. Panel (A) shows sie distribution by number. Panel (B) shows correlation coefficients of the measurements indicating their suitability.

FIG. 7 represents TEM pictures of some of the obtained structures during PISA in DMSO at 15% solid contents of PEG₁₂₅-PMET_{X} with varying hydrophobic block lengths (x).

FIG. 8 represents TEM pictures of some of the obtained structures during PISA after dialysis against water, at 15% solid contents of PEG₁₂₅-PMET_{X} with varying hydrophobic block lengths (x).

FIG. 9 shows a phase diagram in water constructed for PEG₁₂₅-PMet_{X} by systematic variation of the mean target degree of polymerisation of PMet (x) and the total solids concentration used for each synthesis. The mean DP values of the PMET block shown in the phase diagram were calculated from the diblock copolymer composition determined by 1H-NMR spectroscopy in TFA-d.

FIG. 10 shows stimuli-responsive behaviour of PEG₁₂₅-PMet₈₀ vesicles in the presence of reactive oxygen species (ROS) as per Example 3. Panel (A) shows a turbidity assay where light scattering from the nanoparticles was monitored by UV-Vis at 500 nm and found to decrease upon addition of 1 wt % H₂O₂, confirming nanoparticle disassembly. Panel (B) shows a picture of the nanoparticle dispersion both before and 2 hours after addition of 1 wt % H₂O₂. Panels (C), (D) and (E) show dynamic light scattering measurements of nanoparticles before and 2 hours after addition of 1 wt % H₂O₂—panel (C) shows size distribution by number, panel (D) shows size distribution by intensity and panel (E) shows correlograms.

FIG. 11 shows ¹H-NMR spectra in TFA-d (A) of a sample of poly(ethylene glycol)-polymethionine before and after oxidation with H₂O₂ to methionine oxide, as per Example 3.

FIG. 12 shows some gels in DMSO after polymerisation-induced self-assembly according to Example 2. A) From left to right, gels formed by PEG₁₂₅-PMET₅ at different solid content (30, 45 and 60). B) From left to right, gels formed by PEG₁₂₅-PMET₄₀ at different solid content (30, 45 and 60).

FIG. 13 shows an example of a hydrogel in aqueous solution obtained after polymerisation-induced self-assembly and subsequent dialysis of the sample against water according to Example 2.

### DETAILED DESCRIPTION

Described herein is a method for amino acid N-carboxyanhydride (NCA) Polymerization Induced Self-Assembly (also referred to herein simply as “NISA”). A variety of well-defined polypeptide-based amphiphiles have been prepared from natural/proteinogenic (e.g., glutamic acid, lysine, methionine, phenylalanine, histidine) and non-proteinogenic amino acids (e.g., N-substituted amino acids such as sarcosine) using different hydrophilic/hydrophobic combinations and ratios. The supramolecular organization of the structures during polymerization provides an array of nanoparticles that can be used in many biological applications (e.g., as carriers for drug/peptide/protein delivery or imaging agents). By way of example, the inventors have obtained biocompatible and biodegradable polypeptide-based nanoparticles of 150 nm according to dynamic light scattering techniques and transmission electron microscopy. Among the blocks chosen to exemplify the methods and products of the present invention, the inventors have included methionine-based blocks as redox stimuli-responsive block as well as histidine derivative blocks as pH stimuli-responsive blocks. Examples of antifouling and protein repellent blocks aiming for minimal protein interaction and long circulating times, used in this study include polyethylene glycol of different sizes, polysarcosine blocks as well as random KE (lysine-glutamic blocks), PVP, and PMPC. The resulting polypeptide-based nanoparticles offer unique advantages such as: (i) use of well-defined biodegradable polypeptides increases the safety of the system, (ii) the synthetic approach outlined allows for preparation of polypeptide-based nanoparticles in a one-pot reaction configuration; (iii) production at relatively high solids content is possible; (iv) capacity to accommodate several drugs/active agents, allowing combination therapy; and (v) the methods described are readily amenable to industrial scale-up with no additional processing required after production. This makes the NISA-produced nanoparticles an ideal platform for designing therapeutic treatments and diagnostic tools for diseases with unmet medical needs.

The present invention is now described in more detail.

Method of Preparing Self-Assembled Nanoparticles or Microparticles

The method of the invention involves the preparation of self-assembled nanoparticles or microparticles comprise amphiphilic copolymers each comprising a hydrophilic polymer block and a hydrophobic polymer block. The hydrophobic comprises a polypeptide or polypeptoid.

Typically, in the method of the invention hydrophilic polymer blocks are initially provided in a polar aprotic solvent. These hydrophilic polymer blocks correspond to the hydrophilic polymer blocks that, as a result of the synthetic method, are comprised in the amphiphilic copolymers comprised by the nanoparticles or microparticles. The polar aprotic solvent is typically non-aqueous.

The hydrophilic polymer blocks are typically soluble in and/or miscible with the polar aprotic solvent. On the other hand, preferably the hydrophobic polymer block precursor monomers are soluble in and/or miscible with the polar aprotic solvent and said hydrophobic polymer blocks are insoluble in and/or immiscible with the polar aprotic solvent. While there is no particular limitation on the identity of the polar aprotic solvent, representative examples thereof include dimethyl sulfoxide, tetrahydrofuran, dioxane, N,N-dimethyl formamide, N,N-dimethyl acetamide and 1,3-dimethyl-2-imidazolidinone. One exemplary solvent is dimethyl sulfoxide (DMSO). The content of the solvent is not particularly limited, but may, for instance, be from 10-99.9 wt % with respect to the total weight of solvent, hydrophilic polymer blocks and hydrophobic polymer block precursor monomers prior the start of the reaction in step (ii).

In the method, the hydrophilic polymer blocks function as initiator molecules for the polymerization reaction. The hydrophobic polymer block precursor monomers polymerize to produce the hydrophobic polymer block, with polymerization being initiated at the hydrophilic polymer blocks. Thus, the developing hydrophobic polymer block grows onto the hydrophilic polymer blocks. There is no particular limitation on the type of polymerization, which includes polymerization by nucleophilic substitution reaction and by radical propagation, for instance. The amphiphilic polymer hence grows progressively in length, starting from the hydrophilic polymer blocks and increasing in molecular weight as further hydrophobic polymer block precursor monomers become attached to the growing polymer chain.

Advantageously in the method of the invention, the resulting amphiphilic copolymers self-assemble in situ, i.e. in the reaction medium containing the polar aprotic solvent and in which the polymerization has been effected. Thus, preparation of self-assembled nanoparticles or microparticles is achieved in a one-pot reaction, and without the requirement for a separate stage of converting a dilute mixture of amphiphilic polymers to nanoparticles via separate methods (e.g. by film rehydration methods such as those often used in the art).

The resulting self-assembled nanoparticles or microparticles are initially present (e.g., dispersed) in the polar aprotic solvent in which the polymerization and in situ self-assembly was effected. If desired, the self-assembled nanoparticles or microparticles can readily be transferred into an aqueous medium by displacement of the polar aprotic solvent. Routine methods for achieving such transfer to aqueous medium include membrane dialysis, ultrafiltration, size exclusion chromatography or tangential flow filtration. Thus, for instance, an aqueous dispersion of the self-assembled nanoparticles or microparticles can be prepared. The aqueous medium may consist of water, or may comprise water together with any suitable diluent, excipient (e.g., aqueous buffers, preservatives, etc.), and/or additional agent (e.g., a biologically active agent such as a drug).

In a preferred embodiment, the hydrophobic polymer block precursor monomers are cyclic monomers that are susceptible to ring-opening polymerization (ROP) reactions. In such embodiments, the method of the invention thus involves ring-opening polymerization conditions initiated at a suitable functional group on the hydrophilic polymer block and then progressing step-wise by continuing ring-opening reactions along the growing hydrophobic polymer block.

Ring-opening polymerization reactions of amino acids and their derivatives are well known in the art. In particular, such polymerization reactions can be conducted using amino acid N-carboxyanhydrides (also known simply as “NCAs”). The use of NCAs in polypeptide synthesis is well known, would be familiar to those skilled in the art, and so does not require detailed explanation in this document. For instance, a detailed review on NCA polypeptide synthesis is provided in Cheng and Deming, (2011) “Synthesis of Polypeptides by Ring-Opening Polymerization of α-Amino Acid N-Carboxyanhydrides”. In: Deming T. (eds) Peptide-Based Materials. Topics in Current Chemistry, vol 310. Springer, Berlin, the contents of which are herein incorporated by reference in their entirety.

For the avoidance of doubt, the methods of the invention also embrace the use of reagents that are capable of forming NCAs in situ, such as the urethane compounds described elsewhere herein. Thus, the step in the method of the invention of contacting the hydrophilic polymer blocks with hydrophobic polymer block precursor monomers includes a method in which the hydrophobic polymer block precursor monomers are generated in situ from a precursor molecule and thereby come into contact with the hydrophilic polymer blocks.

In one embodiment of the method of the present invention, the hydrophilic polymer blocks are prepared in advance, e.g. from suitable hydrophilic polymer block precursor monomers. Thus, for instance, the polymerization reactions occurring in the one-pot method of the invention may consist in polymerization of the hydrophobic polymer block precursor monomers. Alternatively, however, the method may comprise providing the hydrophilic polymer blocks in situ by polymerizing hydrophilic polymer block precursor monomers. For instance, the one-pot method of the invention may comprise first preparing hydrophilic polymer blocks in the solvent from suitable hydrophilic polymer block precursor monomers, to thereby provide the hydrophilic polymer blocks, and thereafter contacting the hydrophobic polymer block precursor monomers with the resulting hydrophilic polymer blocks. Typically in such a process both polymerization steps are carried out in a single reactor (and e.g., without isolating/purifying intermediates), i.e. the method of the invention remains a one-pot method.

By “one-pot method” or “one-pot process” is meant that the relevant chemical transformations (e.g., reacting the hydrophobic polymer block precursor monomers with the hydrophilic polymer blocks, self-assembly of the amphiphilic polymers, and optionally the preliminary preparation of hydrophilic polymer blocks by polymerization of hydrophilic polymer block precursor monomers) are carried out in a single reaction vessel, typically without removing reaction intermediates from the said single reaction vessel (e.g., without purification or isolation of reaction intermediates). The one-pot nature of the method of the invention is advantageous over methods for preparing self-assembled amphiphilic polymers structure that involve separate reaction stages to effect self-assembly after preparation of the constituent amphiphilic polymers.

Self-Assembled Structures

The self-assembled nanoparticles and microparticles of the present invention can assume any of a number of structures that are known in the art to be formed from amphiphilic copolymers. Non-limiting examples of such structures include vesicles (of any shape, including spherical and/or tubular) and micelles (of any shape, including spherical and/or tubular, the latter also being referred to interchangeably herein as micellar worms or simply “worms”), inverted micelles and planar bilayers. The self-assembled nanoparticles and microparticles can also form gels, for instance via growth of extended micellar worms until gelation results. Conditions such as reaction time, monomer structure, and degree of polymerization can be adjusted to achieve desired product structures.

A “nanoparticle”, as defined herein, is any particle having a smallest end-to-end diameter of between 1 and 100 nm in size. A “microparticle”, as defined herein, is typically any particle having a smallest end-to-end between 0.1 and 100 μm in size. Typically, in a composition comprising a plurality of particles, the relevant diameter is the number average diameter. Typically, particle size is measured using transmission electron microscopy (TEM). Typically, particle size distribution is measured using dynamic light scattering (DLS).

In one preferred embodiment, the nanoparticle or microparticle of the invention is a vesicle, also referred to interchangeably herein as a polymersome. Polymersomes are synthetic vesicles formed from amphiphilic block copolymers. Examples of polymersomes are described in US 2010/0003336 A1, WO 2017/144849, WO 2017/158382, WO 2017/199023 and WO 2017/191444, the contents of each of which are herein incorporated by reference in their entirety. Over the last fifteen years they have attracted significant research attention as versatile carriers because of their colloidal stability, tuneable membrane properties and ability in encapsulating or integrating other molecules (for one representative review article, see J Control Release 2012 161(2) 473-83, the contents of which are herein incorporated by reference in their entirety).

Polymersomes are typically self-assembled structures. Polymersomes typically comprise an amphiphilic block copolymer, i.e. a block copolymer that comprises a hydrophilic block and a hydrophobic block. For example, the polymersome may comprise at least two such amphiphilic block copolymers, which are different from one another.

Such copolymers are able to mimic biological phospholipids. Molecular weights of these polymers are much higher than naturally-occurring phospholipid-based surfactants such that they can assemble into more entangled membranes (J. Am. Chem. Soc. 2005, 127, 8757, the contents of which are herein incorporated by reference in their entirety), providing a final structure with improved mechanical properties and colloidal stability. Furthermore, the flexible nature of the copolymer synthesis allows the application of different compositions and functionalities over a wide range of molecular weights and consequently of membrane thicknesses. Thus the use of these block copolymers as delivery vehicles offers significant advantages.

Polymersomes are often substantially spherical, but can also assume other shapes such as tubular shapes. Polymersomes typically comprise an amphiphilic membrane. The membrane is generally formed from two monolayers of amphiphilic molecules, which align and entangle to form an enclosed core with hydrophilic head groups facing the core and the exterior of the vesicle, and hydrophilic tail groups forming the interior of the membrane.

The thickness of the bilayer is generally between 2 and 100 nm, more typically between 2 and 50 nm (for instance between 5 and 20 nm). These dimensions can routinely be measured, for example by using Transmission Electron Microscopy (TEM) and/or and Small Angle X-ray Scattering (SAXS) (see, for example, J. Am. Chem. Soc. 2005, 127, 8757, the contents of which are herein incorporated by reference in their entirety).

When a polymersome is formed from more than one different type of copolymer, different regions of the polymersome typically have different bilayer thicknesses. For example, if a polymersome is formed from two different types of copolymer, preferably the thickness of the polymersome bilayer of a first region is from 1 to 10 nm, more preferably from 2 to 5 nm. Preferably the thickness of the polymersome bilayer of a second region is from 5 to 50 nm, for instance from 10 to 40 nm. More preferably the thickness of the polymersome bilayer of the second region is from 5 to 20 nm. Preferably the thickness of the polymersome bilayer of the first region is less than the thickness of the polymersome bilayer of the second region. Alternatively, the copolymers can have same thickness but different chemical compositions, which in turn create two different permeabilities with one copolymer forming a bilayer which is less permeable than the other.

In aqueous solution, normally an equilibrium exists between different types of structures, for instance between polymersomes and micelles. In aspects of the invention aimed at the preparation of polymersomes, it is preferred that at least 80%, more preferably at least 90% or 95% by weight and most preferably all of the structures in solution are present as polymersomes. This can be achieved using the methods outlined herein.

It is known that when two different polymersome-forming copolymers are mixed to form a hybrid vesicle they phase-separate and thus give rise to polymersomes that contain discrete regions corresponding to the discrete copolymers. For example, this phenomenon is described in detail in ACS NANO, 5(3), 1775-1784 2011, the content of which is herein incorporated by reference in its entirety. Polymersomes can be readily manufactured by applying these known synthetic principles.

In an alternative embodiment, the nanoparticle or microparticle of the invention is a micelle. Micelles typically comprise a membrane that is generally formed from a single monolayer of amphiphilic molecules, which align and entangle to form an enclosed core of hydrophilic tail groups and with hydrophilic head groups facing the exterior of the micelle. In one aspect, the micelles may be generally spherical in structure. In another aspect, they may become elongated along an axis to form tubular structure known as “worms”. As the worms increase in length, the structures tend to form gels.

One particularly useful characteristic of polymersomes and micelles is their ability to harbour an encapsulated drug. Such a structure is preferably capable of dissociating and releasing an encapsulated drug once it has reached the tissue of interest (i.e. the target tissue). Non-limiting, exemplary tissues of interest include cells (e.g. CNS cells) beyond the blood-brain barrier, immune cells and cancer cells. Preferably the polymersome (or micelle) is capable of dissociating and releasing the encapsulated drug after it has been internalised, via endocytosis, within a target cell (e.g. a CNS cell, an immune cell or a cancer cell).

Dissociation may be promoted by a variety of mechanisms, such as pH sensitivity of the block copolymer, thermal sensitivity of the block copolymer, hydrolysis (i.e. water sensitivity of the block copolymer) and/or redox sensitivity of the block copolymer.

The block copolymer may be a simple A-B block copolymer, or may be an A-B-A or B-A-B block linear triblock copolymer or a (A)₂B or A(B)₂ star copolymers (where A is the hydrophilic block and B is the hydrophobic block). It may also be an A-B-C, A-C-B or B-A-C block linear triblock copolymers or a ABC star copolymers (blocks linked together by the same end), where C is a different type of block. C blocks may, for instance, comprise functional, e.g. cross-linking or ionic groups, to allow for reactions of the copolymer, for instance in the novel compositions. Crosslinking reactions especially of A-C-B type copolymers, may confer useful stability on polymersomes. Cross-linking may be covalent, or sometimes, electrostatic in nature. Cross-linking may involve addition of a separate reagent to link functional groups, such as using a difunctional alkylating agent to link two amino groups. The block copolymer may alternatively be a star type molecule with hydrophilic or hydrophobic core, or may be a comb polymer having a hydrophilic backbone (block) and hydrophobic pendant blocks or vice versa. Such polymers may be formed for instance by the random copolymerisation of monounsaturated macromers and monomers.

When polymersomes (or micelles) comprising encapsulated substances such as drugs are desired, UV spectroscopy and HPLC chromatography may be used to calculate the encapsulation efficiency, using techniques well known in the art. One method of encapsulating a substance such as a drug is to include the drug in the reaction mixture in which the polymerization of hydrophobic polymer block precursor monomers and attendant self-assembly is occurring. An alternative method may involve simple electroporation of the material and polymersomes (or micelles) in water. For instance the drug may be contacted in solid form with an aqueous dispersion of polymersomes (or micelles) and an electric field applied to allow the formation of pores on the polymersome (or micelle) membrane. The solubilised material molecules may then enter the polymersomes (or micelles) though the pores. This is followed by membrane self-healing process with the consecutive entrapment of the material molecules inside the polymersomes (or micelles). Alternatively, material dissolved in organic solvent may be emulsified into an aqueous dispersion of polymersomes (or micelles), whereby solvent and the material become incorporated into the core of the polymersomes (or micelles), followed by evaporation of solvent from the system.

For example, 0.01% to 10% (w/w) of material to be encapsulated is mixed with copolymer in the methods described above.

It is possible to adjust the method of the invention to achieve nanoparticle/microparticles structures of a desired type by controlling factors such as the duration of reaction, and hence degree of polymerization of the hydrophilic and, particularly, hydrophobic blocks, and solids content in the reaction mixture (i.e., the content of self-assembled particles relative to solvent, related to the concentration of polymerizable compounds included in the reaction mixture). Adjusting the ratio of degree of polymerization of the hydrophilic to hydrophobic block can also be used to promote production of the desired structures. For instance, a ratio of degree of polymerization of the hydrophilic to hydrophobic block in the range 1:2.5 to 1:8 may promote vesicle rather than micelle formation.

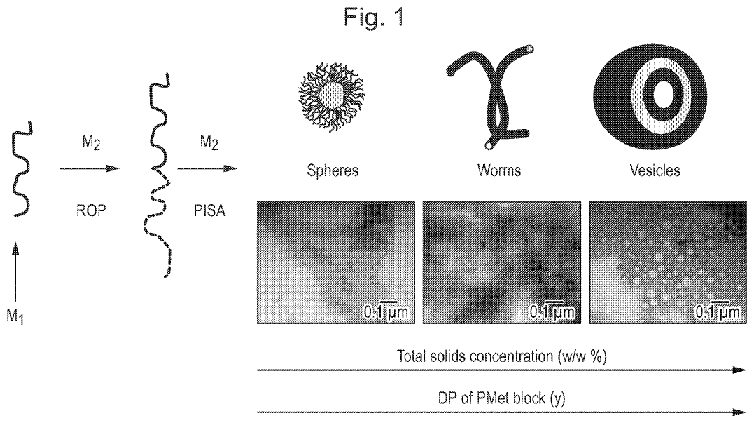

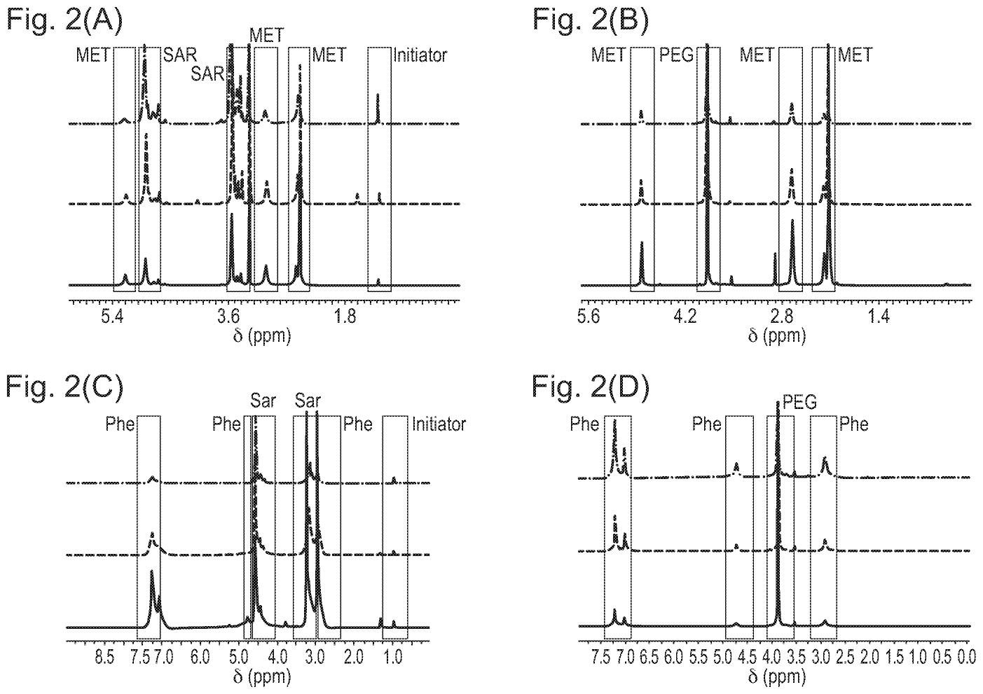

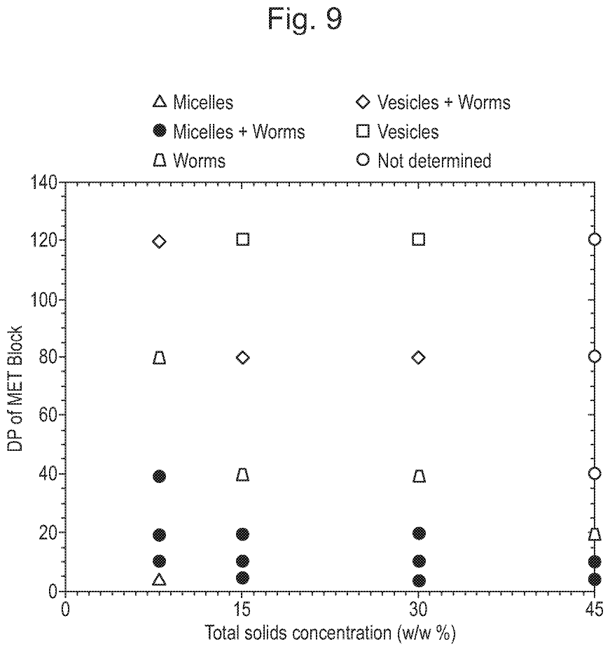

FIGS. 1 and 9 of the present application, as well as Table 5, clearly demonstrate how the reaction conditions can be adjusted to generate the desired self-assembled products. Thus, FIG. 1 shows schematically how increasing the total solids concentration and/or degree of polymerization of the hydrophobic polymer block promotes a transition from sphere/micelle formation, through worm formation, to vesicle formation. Table 5 and FIG. 9 further demonstrate the principles in the context of a specific combination of macroinitiator (hydrophilic block), hydrophobic monomers, solvent, etc (see Example 2 for further details). Again, micelles, then worms, typically are formed at low DP and/or solids concentration, with a transition occurring to vesicle formation at higher combinations of DP and solids concentration. Gels also then begin to emerge at high DP and, especially, solids concentration.

Thus, Example 2 of this application provides a demonstrate of how reaction conditions can readily be optimized, for a specific combination of hydrophilic polymer blocks and hydrophobic polymer block precursor monomers, to obtain each of worm-like micelles, vesicles, and gels. Analogous methods can readily be carried out to achieve desired structures for any combination of amphiphilic copolymer components.

In one aspect of the method of the invention, the reaction steps are terminated when the self-assembled nanoparticles or microparticles constitute from 0.1 to 90% by weight of the total weight of the reaction medium (i.e., the solids content is 0.1 to 90 wt %). More preferably the reaction steps are terminated when the self-assembled nanoparticles or microparticles constitute from 5 to 70% by weight of the total weight of the reaction medium, for instance from 8 to 60% by weight.

In one preferred aspect of the method of the invention, the self-assembled nanoparticles or microparticles comprise vesicles.

In an alternative preferred aspect of the method of the invention, the self-assembled nanoparticles or microparticles comprise micelles. For instance the micelles may be worm-like micelles. In one preferred aspect, the micelles collectively form a gel.

Hydrophilic Polymer Blocks

The hydrophilic polymer block is not particularly limited and can, in general, be constituted from any hydrophilic polymerizable monomer such as those well known and widely used in the art for producing amphiphilic polymers, e.g. for use in constructing micelles and/or polymersomes. A wide variety of suitable such hydrophilic polymers blocks are described in documents such as US 2010/0003336 A1, WO 2017/144849, WO 2017/158382, WO 2017/199023 and WO 2017/191444, the contents of each of which are herein incorporated by reference in their entirety.

By way of example, the hydrophilic block may be based on condensation polymers, such as polyesters, polyamides, polyanhydrides, polyurethanes, polyethers (including polyalkylene glycols, especially polyethylene glycol (PEG)), polyimines, polypeptides (e.g., polysarcosine), polypeptoids, polyureas, polyacetals and polysaccharides. Preferably, the hydrophilic block is based on a polymer selected from a poly(alkylene glycol), poly(vinyl pyrrolidone) (PVP), poly(2-methacryloyloxyethyl phosphorylcholine) (PMPC), poly(glycerol)s, poly(amino acid)s, polysarcosine, poly(2-oxazoline)s, poly[oligo(ethylene glycol) methyl methacrylate] and poly(N-(2-hydroxypropyl)methacrylamide). Most preferably, the hydrophilic block is based on PEG, poly(propylene glycol) or poly[oligo(ethylene glycol) methyl methacrylate]. The hydrophilic block may have zwitterionic pendant groups, in which case the zwitterionic pendant groups may be present in the monomers and remain unchanged in the polymerisation process. It is alternatively possible to derivatise a functional pendant group of a monomer to render it zwitterionic after polymerisation. The hydrophilic block may comprise one polymer or a range of different polymers.

Amphiphilic polymers of the present invention may comprise any of the structural and/or functional features of the polymersomes described in any of WO 2017/144849, WO 2017/158382, WO 2017/199023 and WO 2017/191444, the contents of each of which are herein incorporated by reference in their entirety.

Preferably, the degree of polymerisation of the hydrophilic block is at least 5, preferably at least 10, and more preferably at least 20. Preferably, the degree of polymerisation of the hydrophilic block is at most 200, more preferably at most 175 and more preferably still at most 150. A preferred degree of polymerization of the hydrophilic block is thus 5 to 200, more preferably 10 to 175 and more preferably still 20 to 150.

Hydrophobic Polymer Blocks

In the present invention, the hydrophobic polymer blocks comprise a polypeptide or polypeptoid. A polypeptide is a polymer block comprising or consisting of amino acid residues. A polypeptoid is a block comprising of consisting of N-substituted glycine residues.

As is very well known in the art, an amino acid is a compound that comprises at least one amine functional group and at least one carboxylic acid functional group (i.e., a group of formula —CO2H). Usually the amino acid is an α-amino acid, although an amino acid can also, for example, be a β-amino acid or a γ-amino acid or a δ-amino acid. The amino acid may be naturally occuring, synthetic, proteogenic, or nonproteogenic.

As is well known, many amino acids have chiral centres. It is not important for the purposes of the invention whether the amino acid is chiral or achiral, or whether it is present in a particular enantiomeric form.

Examples of α-amino acids include the well known 20 standard amino acids (specifically, Ala, Cys, Asp, Glu, Phe, Gly, His, Ile, Lys, Leu, Met, Asn, Pro, Gln, Arg, Ser, Thr, Val, Trp and Tyr) as well as other proteinogenic amino acids (e.g. fMet, Sec, Pyl) and also non-proteinogenic amino acids. The amino acid is most often an L-amino acid but may also be a D-amino acid. An amino acid may comprise a chemical modification, including but not limited to a natural post-translation modification.

In general, α-amino acid residues that can be present in the polypeptide may have the general formula —OC(═O)—CH(R)—NH— wherein R is an organic side group (substituent). There is no particularly limitation on the chemical structure of the group R, which may or may not correspond to the side group of the standard and/or proteinogenic acids but may also correspond to that of known non-natural amino acids or other groups, e.g. pendant groups such as those described elsewhere herein. Those skilled in the art would readily appreciate that the invention can routinely be carried out with a wide range of amino acids and could readily implement the principles of the invention to prepare amphiphilic copolymers having polypeptide polymer blocks composed of any amino acids.

Notwithstanding the generality of the invention with respect to applicable polypeptides, non-limiting examples of preferred polypeptides are those comprising amino acid residues selected from the group consisting of methionine, histidine, lysine, glutamic acid, phenylalanine and derivatives thereof. Derivatives of these amino acid residues are not limited, but include, for instance, derivatives of lysine and glutamic acid that incorporate an imidazolyl substituent, and derivatives of glutamic acid in which the carboxylic acid side group is protected (e.g. esterified).

In one particularly preferred aspect, the polypeptide comprises or consists of methionine residues. As discussed elsewhere herein, nanoparticles or microparticles comprising a polymethionine segment may have particularly useful properties in the context of drug delivery applications.

In another particularly preferred aspect, the polypeptide comprises pendant groups having a pKa in the range 4.0 to 7.5. For instance, such pendant groups can be provided by incorporating amino acid residues that bear such pendant groups. One example of suitable such amino acids are histidine residues, as well as other amino acids functionalised to contain imidazolyl groups (lysine and glutamic acid are particularly amenable to such functionalisation). Other suitable pendant groups are those comprising primary, secondary or tertiary amines, or phosphines.

In a still further preferred aspects, the polypeptide comprises chemically reactive pendant groups suitable for further functionalising the self-assembled nanoparticles or microparticles.

Such groups may be nucleophilic or electrophilic groups. They may be used, for instance, to attach a diverse range of additional functional moieties, including but by no means limited to antibodies (and antigen-binding fragments thereof), drugs, imaging agents/labels, additional polymers, and so on.

N-substituted glycine residues can be any substance derived from glycine residues by substitution of its N-atom hydrogen substituent with an organic substituent R. Polypeptoids are known analogues of polypeptides which can provide complementary properties thereto.

In general, N-substituted glycine residues that can be present in the polypeptoid may have the general formula —OC(═O)—CH₂—NR— wherein R is an organic side group (substituent). As with the amino acid residues discussed above, there is no particularly limitation on the chemical structure of the group R. Those skilled in the art would readily appreciate that the invention can routinely be carried out with a wide range of polypeptoids and could readily implement the principles of the invention to prepare amphiphilic copolymers having polypeptoid polymer blocks composed of any N-substituted glycine residues.

As discussed elsewhere herein, polymerization in the method of the invention can in a preferred embodiment be effected by ring-opening polymerization reactions using amino acid N-carboxyanhydrides. In the context of the non-limiting α-amino acid and N-substituted glycine residues described above, such cyclic N-carboxyanhydride reagents may have the structure

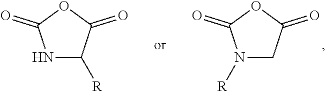

respectively.

In one embodiment of the method of the invention, step (ii) comprises providing the NCAs as reagents, i.e. a physical step of providing the NCAs and mixing them with the hydrophilic polymer blocks (or the hydrophilic polymer block precursor monomers where the hydrophilic polymer blocks are to be formed in situ).

Alternatively, as is known in the art, NCAs are capable of forming in situ from suitably activated amino acids (or N-substituted glycines). Thus, step (ii) of the method may comprise allowing the hydrophilic polymer blocks to contact hydrophobic polymer block precursor monomers (specifically NCAs or N-substituted glycines) formed in situ from activated amino acid molecules. In this case, the method may comprise a physical step, prior to step (ii), of providing the activated amino acid (or or N-substituted glycines) molecules and mixing them with the hydrophilic polymer blocks (or the hydrophilic polymer block precursor monomers where the hydrophilic polymer blocks are to be formed in situ).

There is no particular limitation on the structure of such activated amino acids or N-substituted glycines, provided that they are capable of forming corresponding NCAs in situ. However, in a preferred embodiment the activated molecules are urethane derivatives of the corresponding amino acid or N-substituted glycine, i.e. in which a hydrogen attached to the (non-side chain) amino group of the relevant amino acid is replaced by an activating group that transforms the amino group into a urethane group (i.e. —C(O)—O—R′ replaces the —H that is attached to the amino group in the relevant amino acid; R′ can be any suitable substituent, including but not limited to halogen atoms and organic and hydrocarbyl groups). Such urethane derivatives of amino acids and N-substituted glycines are known in the art to be susceptible to cyclization to form NCAs under mild conditions (see, e.g., Doriti, Polym. Chem., 2016, 7, 3067-3070).

Preferably, the degree of polymerisation of the hydrophobic block is at least 5, preferably at least 10, and more preferably at least 20. Preferably, the degree of polymerisation of the hydrophobic block is at most 400, more preferably at most 300, more preferably still at most 200 and most preferably at most 150. A preferred degree of polymerization of the hydrophobic block is thus 5 to 400, more preferably 10 to 300 or 10 to 200 and more preferably still 20 to 150.

Hydrogels

The present invention also extends to hydrogels comprising a plurality of micellar nanoparticles or microparticles, wherein said nanoparticles or microparticles each comprise amphiphilic copolymers each comprising a hydrophilic polymer block and a hydrophobic polymer block that comprises a polypeptide or polypeptoid. Such hydrogels can beneficially be made using the methods of the present invention. They may comprise entangled worm-like micelles, which can trap water, together with other substances of interest.

The hydrogels can be used without limitation for any known application of hydrogels, including but by no means limited to preparation of delivery agents, as adjuvants in vaccines, and in tissue engineering applications.

Vesicles

Still further, the present invention extends to a vesicle that comprises an amphiphilic block copolymer comprising a hydrophilic block and a hydrophobic block that comprises a polypeptide or polypeptoid, wherein said vesicle is suitable for administration to a subject and said hydrophobic block is capable of undergoing a chemical transformation, leading to degradation of said vesicle in vivo, in response to a change in in vivo conditions. The ability of the vesicle to degrade in this way may be of significant utility in drug delivery applications, e.g. where a drug is encapsulated within the vesicle and/or attached to the vesicle surface. Such vesicles can, for instance, be advantageously obtained using the methods of the present invention.

The relevant change in in vivo conditions can be any of a range of phenomena, including a change in pH (for instance, associated with the process of endocytosis), a change in concentration of reactive oxygen species, ROS (for instance, high concentrations of ROS may be present at a disease site), or a change in temperature.

In one such embodiment, the change in in vivo conditions is an increase in the concentration of reactive oxygen species (ROS). In this embodiment, an exemplary component of the hydrophobic block is methionine residues. As is discussed in more detail in Example 3, methionine residues are sensitive to ROS and may promote the degradation of the vesicles in regions of high ROS.

In another embodiment, the change in in vivo conditions is a change in pH and the hydrophobic block comprises pendant groups having a pKa in the range 4.0 to 7.5. Examples of hydrophobic blocks comprising such pendant groups are described elsewhere herein. In one embodiment, for instance, the pendant groups may comprise imidazolyl groups and preferably at least some of these imidazolyl groups are provided by histidine residues in the hydrophobic block.

Encapsulated or Surface-Attached Drugs

The nanoparticle or microparticle may comprise a drug encapsulated within the nanoparticle or microparticle. It is also possible to encapsulate a plurality of different drugs within a single nanoparticle or microparticle, or to provide a plurality of nanoparticles or microparticles each containing a particular encapsulated drug.

As will be readily understood, the encapsulated drug is selected in accordance with the disorder to be treated. Non-limiting examples of such disorders are described elsewhere in this disclosure.

Non-limiting examples of drugs include: a drug that is effective for the treatment or prevention of a brain disorder; a drug that is effective for the treatment or prevention of the immune and/or inflammatory disorder; and a drug that is effective for the treatment or prevention of a cancer. There is no particular limitation on the identity of the drug and so drugs can be selected from those known in the art for treatment or prevention of the disorder of interest in any given embodiment.

Non-limiting examples of drugs include neuroprotectants, immunomodulatory drugs (“immunomodulators”), non-steroidal anti-inflammatory drugs (NSAIDs), corticosteroids, disease-modifying antirheumatic drugs (DMARDs) immunosuppressants, TNF-alpha inhibitors and anti-cancer drugs, nucleic acids (double stranded oligonucleotides i.e. DNAs, single stranded oligonucleotides i.e. RNAs) and aptamers (oligonucleotide or peptide molecules).

Illustrative and non-limiting examples of specific drugs that may be encapsulated include fumarate and fumarate esters, glutamate antagonists (e.g., Estrogen, Ginsenoside Rd, Progesterone, Simvastatin, Memantine), antioxidants (e.g., Acetylcysteine, Crocin, Fish oil, Minocycline, Pyrroloquinoline quinone (PQQ), Resveratrol, Vinpocetine, Vitamin E), Stimulants (e.g., Selegiline, Nicotine, Caffeine), Caspase inhibitors, Trophic factors (e.g., CNTF, IGF-1, VEGF, and BDNF), Anti protein aggregation agents (e.g. sodium 4-phenylbutyrate, trehalose, and polyQ-binding peptide), Erythropoietin, Lithium, carnosine, asiatic acid, flavonoids (e.g. xanthohumol, naringenin, galangin, fisetin and baicalin), cannabinoids (e.g., WIN55,212-2, JWH-133 and TAK-937), citicoline, minocycline, cerebrolysin, ginsenosoid-Rd, granulocyte-colony stimulating factor, Tat-NR2B9c, magnesium, albumin, paracetamol, aspirin, choline and magnesium salicylates, celecoxib, diclofenac (e.g. diclofenac potassium, diclofenac sodium), diflunisal, etodolac, fenoprofen, flurbiprofen, ibuprofen, indomethacin, ketoprofen, meclofenamate, mefenamic acid, meloxicam, nabumetone, naproxen (including naproxen sodium), oxaprozin, piroxicam, rofecoxib, salsalate, sodium salicylate, sulindac, tolmetin, valdecoxib, corticosteroids, alemtuzumab, interferon beta-1b, fingolimod, glatiramer acetate, natalizumab, plegridy, peginterferon beta 1a, teriflunomide, methotrexate, sulfasalazine, leflunomide, adalimumab, etanercept, golimumab, ustekinumab, azathioprine, cyclosporine, infliximab, golimumab, certolizumab, hydroxychloroquine, methotrexate, azathioprine, mycophenolate, acitretin, hydrea, isotretinoin, mycophenolate mofetil, sulfasalazine, 6-thioguanine, calcipotriol, calcitriol, tacalcitol, tacrolimus, pimecrolimus, dithranol, endamustine, bendamustine, carmustine, chlorambucil, cyclophosphamide, dacarbazine, ifosfamide, melphalan, procarbazine, streptozocin, temozolomide, capecitabine, 5-Fluoro Uracil, Fludarabine, Gemcitabin, Methotrexate, Pemetrexed, Raltitrexed, Actinomycin D, Bleomycin, Doxorubicin, Epirubicin, Mitomycin, Mitoxantrone, Etoposide, Docetaxel, Irinotecan, Paclitaxel, Topotecan, Vinblastine, Vincristine, Vinorelbine, Eribulin, Carboplatin, Cisplatin, Oxaliplatin, Afatinib, Aflibercept, BCG, Bevacizumab, Brentuximab, Cetuximab, Crizotinib, Denosumab, Erlotinib, Gefitinib, Imatinib, Interferon, Ipilimumab, Lapatinib, Panitumumab, Pertuzumab, Rituximab, Sunitinib, Sorafenib, Trastuzumab emtansine, Temsirolimus, Trastuzumab, Vemurafenib, Clodronate, Ibandronic acid, Pamidronate, Zolendronic acid, Anastrozole, Abiraterone, Bexarotene, Bicalutamide, Buserelin, Cyproterone, Degarelix, Exemestane, Flutamide, Folinic acid, Fulvestrant, Goserelin, Lanreotide, Lenalidomide, Letrozole, Leuprorelin, Medroxyprogesterone, Megestrol, Mesna, Octreotide, Stilboestrol, Tamoxifen and Thalidomide.

Alternatively or additionally, any such drug can also be attached to the surface of nanoparticle or microparticle by any means known in the art, for instance by exploiting reactive functional groups on the surface of the particles.

Pharmaceutical Compositions

The nanoparticles or microparticles can be formulated as a pharmaceutical composition using routine techniques known in the art. For example, pharmaceutical compositions already utilised for the formulation of nanoparticles or microparticles such as polymersomes or drug-containing liposomes.

The pharmaceutical composition comprises a plurality of the nanoparticles or microparticles. It also comprises one or more pharmaceutically acceptable excipients or diluents. The one or more pharmaceutically acceptable excipients or diluents may be any suitable excipients or diluents. The pharmaceutical composition is typically aqueous, i.e. it contains water (in particular sterile water).

A typical pH of the aqueous pharmaceutical composition is 7.0 to 7.6, preferably 7.2 to 7.4. Pharmaceutically acceptable buffers may be used to achieve the required pH. The pharmaceutical composition may be in the form of a sterile, aqueous, isotonic saline solutions.

Typically the pharmaceutical composition is an injectable composition, e.g. it is suitable for intravenous delivery, for example it is suitable for infusion.

Medical Uses of the Nanoparticles or Microparticles

In many aspects, the nanoparticles or microparticles made by the methods of the present invention and/or of the present invention are able to target tissues including, but not limited to cells (e.g. CNS cells) beyond the blood-brain barrier, immune cells and cancer cells and to release drugs once localised at the target. Thus, the nanoparticles or microparticles can be used in methods for the improved targeted treatment of diseases and other pathological conditions. Often in such embodiments the desired drug is encapsulated in, for instance, a vesicle (or micelle) nanoparticle/microparticle. Alternatively or additionally, drugs can be attached to the surface of the nanoparticle/microparticle.

As will be readily understood, suitable drugs are selected in accordance with the disease to be treated. For example, if the disorder is a brain disorder then the drug is a drug that is effective for the treatment or prevention of the brain disorder. If the disorder is an immune and/or inflammatory disorder then the drug is a drug that is effective for the treatment or prevention of the immune and/or inflammatory disorder. If the disorder is a cancer then the drug is a drug that is effective for the treatment or prevention of the cancer. Ligands, for instance attached to the nanoparticle/microparticle surface, can assist in directing the particles selectively towards the tissues of interest.

Examples of brain disorders include stroke, neurodegenerative diseases, traumatic brain injury (TBS), spinal cord injury, and neurotoxin consumption (for example, methamphetamine overdoses). Neurodegenerative diseases include conditions such as amyotrophic lateral sclerosis, Parkinson's disease, Alzheimer's disease and Huntington's disease. Stroke may be ischemic stroke or haemorrhagic stroke.

Examples of immune and/or inflammatory disorders include multiple sclerosis, psoriatic arthritis, rheumatoid arthritis, lupus erythematosus and psoriasis.

Examples of cancers include: cancers of the skin, such as melanoma; lymph node; breast; cervix; uterus; gastrointestinal tract; lung; ovary; prostate; colon; rectum; mouth; brain; head and neck; throat; testes; thyroid; kidney; pancreas; bone; spleen; liver; bladder; larynx; nasal passages; AIDS-related cancers; cancers of the blood and bone marrow, such as multiple myeloma and acute and chronic leukemias, for example, lymphoblastic, myelogenous, lymphocytic, and myelocytic leukemias; advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, chronic lymphocytic leukemia (CLL), Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, eiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

Further disorders that may be susceptible to treatment or prevention with the nanoparticles or microparticles of the invention include HIV, atherosclerosis, ischemic heart disease and obstructive sleep apnoea.

Medical uses and methods of treatment, of course, involve the administration of a therapeutically effective amount of the nanoparticle or microparticle. A therapeutically effective amount of the nanoparticles or microparticles is administered to a patient. A typical dose is from 0.001 to 1000 mg, measured as a weight of the drug, according to the activity of the specific drug, the age, weight and conditions of the subject to be treated, the type and severity of the disease and the frequency and route of administration. Preferably, daily dosage levels are from 0.001 mg to 4000 mg.

The present invention further provides a method of treating or preventing a disorder that comprises administering a therapeutically effective amount of a nanoparticle or microparticle of the invention to a patient in need thereof. For example, the present invention provides a method of treating or preventing a disorder selected from any disorder specified in this disclosure, the drug being a drug that is capable of treating or preventing the said disorder, such as a brain disorder, an immune and/or inflammatory disorder, or a cancer. The present invention still further provides the use of a nanoparticle or microparticle of the present invention in the manufacture of a medicament for use in a method of treating or preventing a disorder as identified above.

The present invention further provides a vaccine comprising a nanoparticle or microparticle of the invention and an antigen. An antigen is any agent that causes the immune system of an animal body to produce an immune response, e.g. bacteria, viruses or pollen. Typically, the vaccine is administered to a human or animal recipient to induce the memory function of the adaptive immune system towards the specific antigen contained in the vaccine. Preferably, the nanoparticles or microparticles in the vaccine bind selectively to dendritic cells. Further preferably, the vaccine is a cancer vaccine.

In all aspects of the present invention, the nanoparticles or microparticles may further comprise a label or imaging agent (e.g., encapsulated therein and/or attached to the surface of the particles). For instance, the label/imaging agent could be a dye. A dye for imaging refers to any substance that is used as a label, or that enhances specific structures in any imaging technique. An imaging agent, hence, includes optical an imaging agent, magnetic resonance imaging agent, radioisotope, and contrast agent. Examples, without limitation, of optical imaging agents are an acridine dye, a coumarin dye, a rhodamine dye, a xanthene dye, a cyanine dye, a pyrene dye, Texas Red, Alexa Fluor® dye, BODIPY® DYE, Fluorescein, Oregon Green® dye, and Rhodamine Green™ dye, which are commercially available or readily prepared by methods known to those skilled in the art. Examples of imaging agents appropriate for the present invention include, but are not limited to, transition metals and radioactive transition metals chelated to chelating agents for instance DTPA (diethylene triamine pentaacetic adic), DOTA (1,4,7,10-tetraazacyclododeane-1,4,7-tetraacetic acid) and NOTA (1,4,7-Triazacyclononane-1,4,7-triacetic acid).

Further, in all aspects of the present invention, the nanoparticles or microparticles may further comprise a targeting unit i.e. antibodies, peptides, proteins etc. (e.g., encapsulated therein and/or attached to the surface of the particles). A targeting unit is any chemical structure that functionally interacts with a binding site to cause a physical association between the agent and a surface, e.g., a cell surface. The term targeting unit embraces any molecule (e.g., a naturally occurring molecule, or a chemically/physically modified variant thereof) that is capable of binding to a binding site on the target surface. The binding site could be, but not exclusively, also be capable of internalisation (eg. endosome formation)—also referred to as receptor-mediated endocytosis. The targeting unit may possess an endosomal membrane translocation function, in which case separate targeting unit and Translocation Domain components need not be present in an agent of the present invention.

### EXAMPLES

The present invention is illustrated by the following examples. However, these examples do not limit the scope of the invention.

### Example 1 (Reference): Polymer Synthesis and Characterisation

Polypeptide-based block-copolymers were synthesised using either the ROP of NCAs method (ring-opening polymerization of N-carboxyanhydrides), or a variation of the method based on the synthesis of urethane-derivatives as more stable and less moisture-sensitive monomers.

The synthesis of Trityl-L-Histidine, Dinitrophenyl (DNP)-L-Histidine urethane derivatives is reported for the first time, whereas the natural and non-natural amino acids Sarcosine, L-Methionine, L-Z-Lysine, L-Benzyl-protected glutamate and L-Phenylalanine urethane derivatives were synthesised according to the literature (Yamada, J. Polymer Science Part A 51(21), 2013, 4565-4571; Doriti, Polym. Chem., 2016, 7, 3067-3070).

Sarcosine was selected to form a hydrophilic block to provide stealth properties due to its well-known protein repellent abilities (which may be useful, for instance, to allow for long in vivo circulation times required to target the disease). Other hydrophilic blocks such as Polyethylene glycol (functionalised or not), PMPC and PVP were also used because of their protein repellent ability.

The other amino-acids were selected to form the hydrophobic blocks. L-Methionine may be particularly useful due to its stimuli-responsive behaviour in reductive media. L-Histidine (or imidazole containing modified lysine/glutamic) may be particularly useful due to its stimuli-responsive properties owing to its biologically relevant pKa of 6.8. y-Benzyl-L-glutamate may be particularly useful due to the possibility of partial deprotection of the carboxylic groups allowing the introduction of crosslinking sites. L-phenylalanine was selected as a non-stimuli responsive control.

Polymerisation was optimised for each particular hydrophobic amino acid and selected degree of polymerisation (DP) in terms of solvent, reaction time, temperature, and addition of co-operative base as catalyst (TEA). The use of the common denaturant urea was also explored for the synthesis of long hydrophobic blocks. Those amphiphiles have been characterised by Nuclear Magnetic Resonance.

Typically, for the particular case of the use of a macroinitiator as a hydrophilic block, compounds were synthesised as follows. The hydrophobic monomer precursor (an active urethane derivative precursor of the relevant amino acid, which cyclises in situ to form an NCA reagent with temperature) of the hydrophobic block and the macro-initiator (hydrophilic block) were weighed in a Schlenk flask provided with a stirrer bar and a suba-seal stopper. The mixture was subjected to three high vacuum/argon cycles to remove residual moisture. Each equivalent depends on the desired hydrophobic/hydrophilic ratio in the final polymer, being typically, 1 the initiator, and x the equivalents of monomer according to the desired hydrophobic length. The reaction was performed using Schlenk techniques under argon atmosphere. The corresponding amount of the aprotic polar solvent (DMSO) according to the desired % w of solid content was then added. The reaction was left then to proceed at 60° C. under vigorous stirring and argon atmosphere with an outlet for CO2 removal for the desired time depending on the required length. The final self-assembled product could be dialysed against the same aprotic solvent to remove possible unreacted monomer or impurities using MWCO 3.5 kDa. For polymer isolation for characterisation, an aliquot could be precipitated in diethyl ether, washed 3× with diethyl ether, dried and analysed using 1H-NMR and GPC.

TABLE 1
Examples of block copolymer characterisation by 1H-NMR.

| | DP hydrophilic | DP hydrophobic | | |
|---|---|---|---|---|
| Polymer | block | block (NMR) | Mn (NMR) Da | Yield (%) |
| PSar100-b-PMet20 | 80 | 21 | 7950 | 83 |
| PSar100-b-PMet40 | 98 | 37 | 11220 | 82 |
| PSar100-b-PMet80 | 99 | 78 | 16650 | 89 |
| N3PSar100-b-PMet80 | 97 | 73 | 15870 | 90 |
| PEG125-b-PMet20 | 125 | 22 | 8380 | 88 |
| PEG125-b-PMet40 | 125 | 45 | 11395 | 89 |
| PEG125-b-PMet80 | 125 | 80 | 15980 | 93 |
| PEG45-b-PMet40 | 45 | 45 | 7875 | 87 |
| N3-PEG125-b-PMet80 | 125 | 80 | 15980 | 90 |
| PEG125-b-Pphe20 | 125 | 22 | 8734 | 78 |
| PEG125-b-Pphe40 | 125 | 41 | 11527 | 69 |
| PEG125-b-Pphe80 | 125 | 73 | 16231 | 60 |
| PSar20-b-PPhe20 | 29 | 20 | 4825 | 78 |
| PSar50-b-PPhe50 | 60 | 41 | 9927 | 85 |

*DP: Degree of polymerisation. Mn: molecular weight estimated by NMR

Batch-to batch reproducibility was consistent between different polymerisation batches as can be observed by the examples summarised in Table 2.

TABLE 2
Examples of PEG125-PMET80 block-copolymer characterisation
by 1H-NMR showing batch to batch reproducibility

| | | DP | DP | | |
|---|---|---|---|---|---|
| | | hydrophilic | hydrophobic | Mn (NMR) | Yield |
| Batch | Polymer | block | block (NMR) | Da | (%) |
| _0055 | PEG125-b-PMet80 | 125 | 80 | 15980 | 93 |
| _0134 | N3-PEG125-b-PMet80 | 125 | 80 | 15980 | 90 |
| _0142 | PEG125-b-PMet80 | 125 | 86 | 16766 | 95 |
| _0145 | PEG125-b-PMet80 | 125 | 89 | 17159 | 97 |
| _0151 | PEG125-b-PMet80 | 125 | 80 | 15980 | 90 |
| _0154 | PEG125-b-PMet20 | 125 | 85 | 16635 | 90 |

### Example 2: Polymerisation Induced Self-assembly

The self-assembly of amphiphilic block copolymers is usually limited to dilute copolymer solutions (<1%), which represents a significant disadvantage for potential commercial applications due to obvious problems in manufacture and scaling up processes.

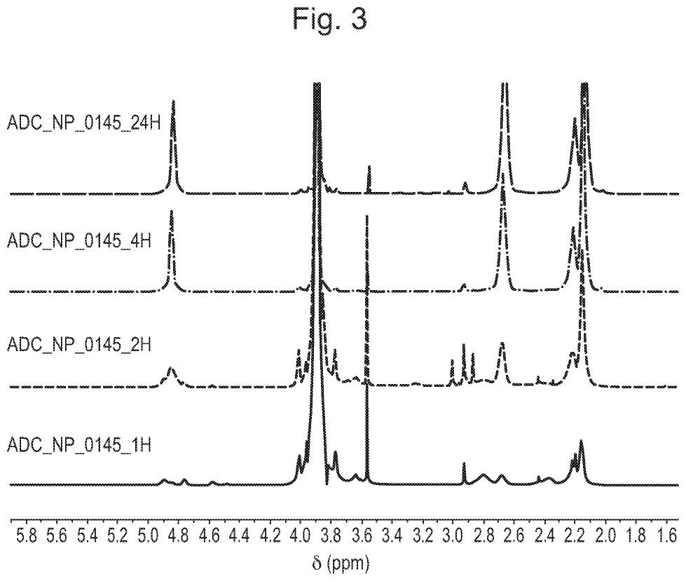

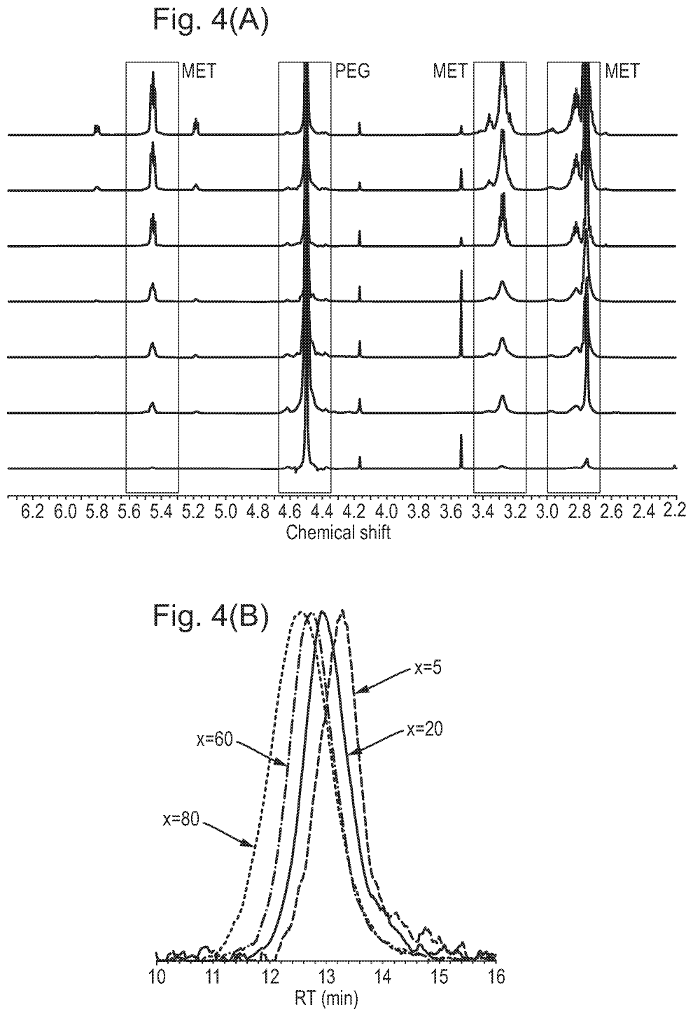

Herein, there is described for the first time a method to produce well-defined nanoparticles by NCA polymerisation Induced Self-Assembly (NISA). Concretely, hydrophobic blocks were polymerised in DMSO (good solvent for the monomer), using the hydrophilic block as macroinitiator. Self-assembly took place during polymerisation and complete reaction was achieved at 4 h (according to NMR, see FIG. 3 and Table 3). General synthetic conditions for the polymerisation were as reported in Example 1.

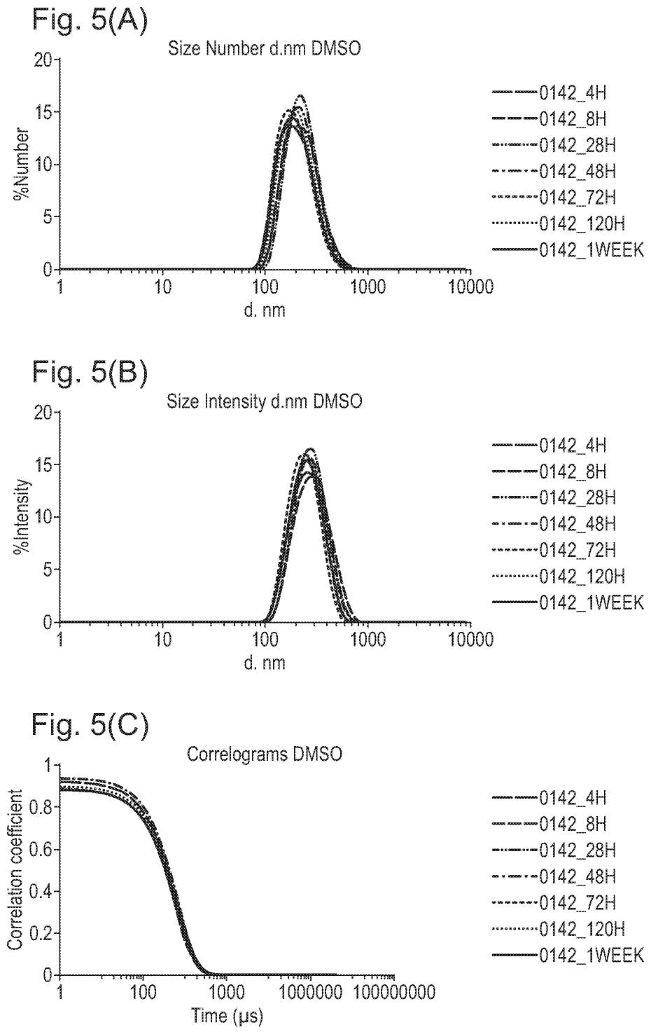

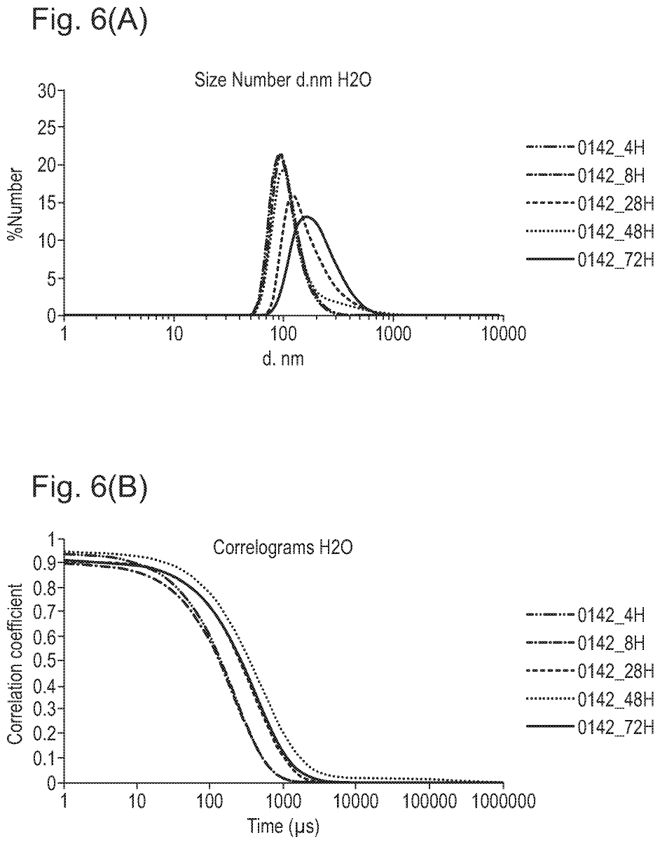

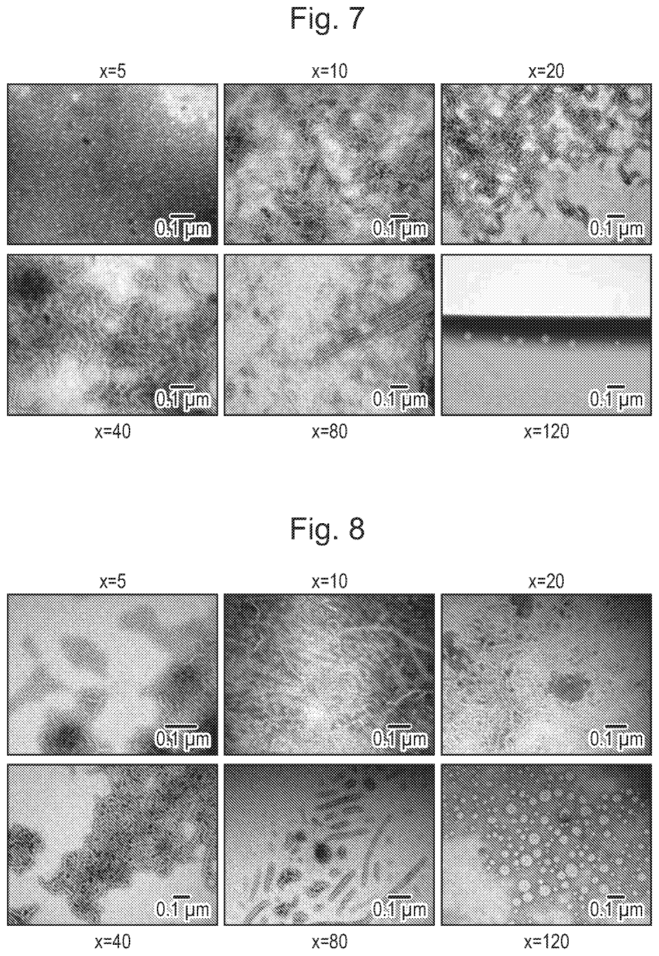

Particle size of the samples, analysed in the polymerisation media (DMSO, see FIG. 5) revealed sizes of around 200 nm (Table 4).

The formed vesicles were then transferred to water via dialysis procedures.

TABLE 3
Example of NISA kinetics characterisation of
PEG₁₂₅ initiated POC-Methionine by ¹H-NMR.

| Time (h) | DP Hydrophobic block (MET) |
|---|---|
| 1 | 8 |
| 2 | 15 |
| 2.5 | 33 |
| 3 | 42 |
| 3.5 | 58 |
| 4 | 80 |
| 8 | 85 |
| 24 | 89 |
| 48 | 92 |
| 72 | 95 |
| 120 | 95 |

TABLE 4
Polymerisation induced self-assembly of Poly(ethylene glycol)-polymethionine.
Particles sizes obtained by dynamic light scattering at 0.5 mg/mL

| | | Number | Intensity | Volume | | Diffusion |
|---|---|---|---|---|---|---|
| Sample | Z-Ave | mean | Mean | Mean | | coefficient |
| name | (d · nm) | (d · nm) | (d · nm) | (d · nm) | Pdl | (μ²/s) |
| 4H_DMSO | 231.8 | 212.6 | 280.6 | 334.4 | 0.208 | 0.948 |
| 8H_DMSO | 255.8 | 231.5 | 305 | 368.8 | 0.171 | 0.859 |
| 28H_DMSO | 252.2 | 242.9 | 290.7 | 335.8 | 0.142 | 0.872 |
| 48H_DMSO | 249.2 | 235.2 | 291 | 340.6 | 0.174 | 0.882 |
| 72H_DMSO | 221.3 | 200.5 | 248.9 | 285.3 | 0.131 | 0.993 |
| 120H_DMSO | 232.7 | 219.5 | 272 | 315.7 | 0.153 | 0.945 |
| 168H_DMSO | 224.2 | 209.9 | 266.7 | 310.7 | 0.18 | 0.98 |

TABLE 5
Physical state observation of different PEG₁₂₅-PMETx
after one-pot polymerisation induced self-assembly.

| Reaction | DP/Solid | | | | | |
|---|---|---|---|---|---|---|
| time | contents w % | 8 | 15 | 30 | 45 | 60 |
| 4 h | 5 | Transp. | Transp. | Gel | Gel | Gel |
| | | liquid | liquid | | | |
| | 10 | Transp. | Transp. | Viscous | Gel | Gel |
| | | liquid | liquid | Transp. | | |
| | | | | liquid | | |
| | 20 | Transp. | Transp. | Gel | Gel | Gel |
| | | liquid | liquid | | | |
| | 40 | Transp. | Viscous | Viscous | Gel | Gel |
| | | liquid | Transp. | Turbid | | |
| | | | liquid | liquid | | |
| 24 h | 60 | Turbid | Turbid | Gel | Gel | Gel |
| | | liquid | liquid | | | |
| | 80 | Turbid | Gel | Gel | Gel | Gel |
| | | viscous | | | | |
| | | liquid | | | | |
| 48 h | 120 | Turbid | Turbid Gel | Gel | Gel | Gel |
| | | viscous | | | | |
| | | liquid | | | | |

TABLE 6
Particles sizes of different batches of PEG₁₂₅-PMET₈₀ polymersomes
obtained after one-pot polymerisation induced self-assembly.

| Sample name | 0142_4 h | 0145_4 h |
|---|---|---|
| Z-Ave (d.nm) | 148 | 142.6 |
| Pdl | 0.126 | 0.137 |
| Diffusion coefficient (μ²/s) | 3.32 | 3.45 |
| Mean Count Rate (kcps) | 152.8 | 188.8 |
| Number Mean (d.nm) | 106.4 | 94.66 |
| Volume Mean (d.nm) | 153 | 145.3 |
| Intensity Mean (d.nm) | 164.5 | 161.8 |

Strategies for Labelling and Bioconjugations

The versatility of the NISA strategy allows one to easily introduce orthogonal functionalities at the nanoparticle surface by using a certain proportion of bifunctional initiators. Those functionalities will ultimately be used for bioconjugation of, for instance, dyes, peptides, drugs or antibodies.

Example of Cy5 Dye Conjugation:

NCA polymerisation induced self-assembly was performed by using a known proportion of N₃-PEG125-NH₂/MeO-PEG125-NH₂. After the reaction, the sample was dialysed against DMSO and direct conjugation of alkyne-Cy5 dye was performed over the purified product by Copper-catalyzed Alkyne Azide cycloaddition (CuAAC). Briefly, all solutions were previously degassed under N₂ flow. The alkyne dye was added to the DMSO solution containing the nanoparticles. Next, CuSO₄ and sodium ascorbate were added in degassed milliQ water reaching a final proportion of DMSO/water.

The particle solution was left under vigorous stirring at under N₂ atmosphere for 72 hours. Labelled particles were then dialyzed against DMSO first and then transferred to milliQ water. % Labelling was calculated by absorbance at 656 nm of a freeze-dried aliquot of the sample. Conjugation efficiency: 90%.

### Example 3. Stimuli-Responsive Behaviour

Methionine Bearing Nanoparticles, Responsive to ROS Stimuli.

Reactive oxygen species (ROS), widely exist in living organisms and play crucial roles in cell signalling pathways. An increased production of ROS in many pathological conditions results in oxidative stress which may disrupt cellular homeostasis, and impair cell components including membrane lipids, proteins, and DNA. Indeed, chronic oxidative stress has been linked to mitochondria disfunction and many pathologies including atherosclerosis, neurodegeneration, arthritis, diabetes, and cancer. The unique redox microenvironments in pathological conditions, make these regions different from their surroundings, and that can be exploited and used to specifically deliver drugs/bioactive agents/imaging agents to the disease site. For instance, H₂O₂ levels (one of ROS) in normal human plasma is in average 3×10⁻⁶ M. In contrast, intracellular concentrations can be in the range of 10-1000×10⁻⁶ M (i.e. activated macrophages).

Methionine is well-known to undergo oxidation under the exposure to common oxidants (including those considered as ROS), yielding first methionine sulfoxide under mild oxidation, and finally methionine sulfone, under harsh oxidation. Both species are hydrophilic, and therefore, the hydrophobic to hydrophilic transition of methionine allows for design of ROS-responsive vesicles. These vesicles are meant to remain stable during circulation in plasma (where ROS levels are low), and disassemble at the disease site (where oxidative stress will lead to high ROS concentrations), as illustrated in the scheme below.

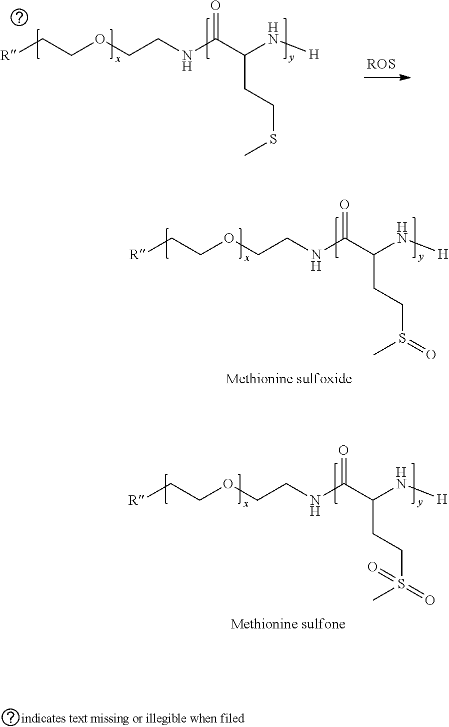

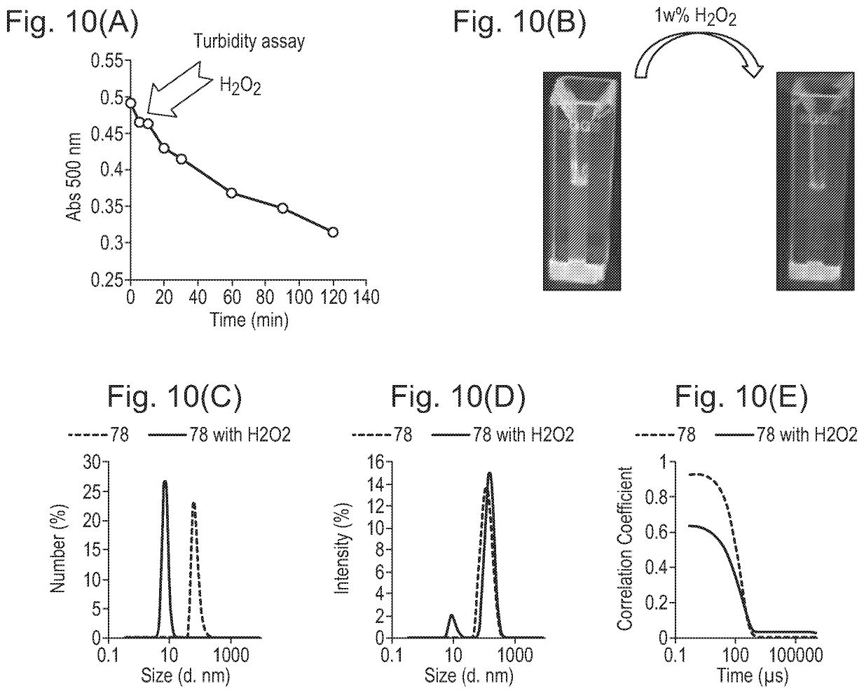

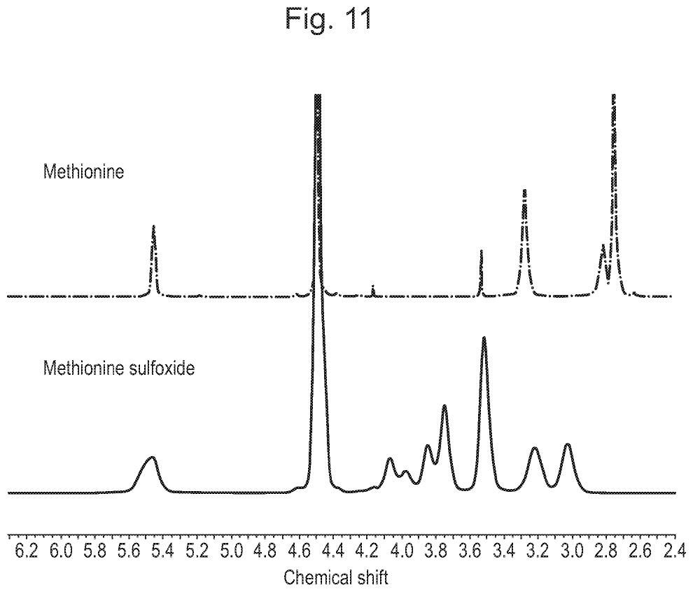

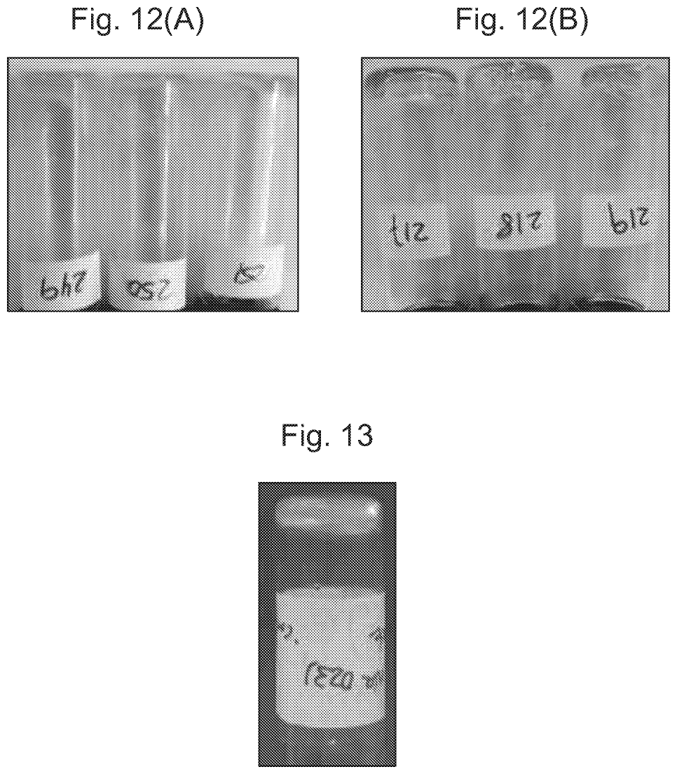

A turbidity essay was conducted in order to demonstrate the stimuli-responsive properties of methionine-based nanostructures generated using the method described herein. Briefly, a sample containing PEG₁₂₅-PMET₈₀ assemblies in aqueous solution was subjected to hydrogen peroxide (1% wt) and the disassembly of those was followed using UV-VIS by means of recording the decrease in the scattering intensity over time (FIG. 10 A). Further confirmation was obtained by dynamic light scattering techniques, where the lack of a good correlation function indicated the disassembly of the system. Moreover, ¹H NMR in TFA-d before and after the oxidation process revealed the complete conversion of methionine (hydrophobic) into methionine oxide (hydrophilic and water soluble) (FIG. 11).

1. A method of preparing self-assembled nanoparticles or microparticles, wherein:
said self-assembled nanoparticles or microparticles comprise amphiphilic copolymers each comprising a hydrophilic polymer block and a hydrophobic polymer block;
said hydrophobic polymer block comprises a polypeptide or polypeptoid; and
said method comprises:
(i) providing, in a polar aprotic solvent, hydrophilic polymer blocks as initiator molecules;
(ii) contacting said hydrophilic polymer blocks with hydrophobic polymer block precursor monomers, and forming said hydrophobic polymer blocks by polymerization reactions, initiated at the hydrophilic polymer blocks, of said hydrophobic polymer block precursor monomers, thereby producing said amphiphilic copolymers; and
(iii) allowing said amphiphilic copolymers to self-assemble in situ to form said self-assembled nanoparticles or microparticles.

2. The method of claim 1, which further comprises transferring said self-assembled nanoparticles or microparticles into an aqueous medium by displacement of the polar aprotic solvent, optionally wherein said transferring into an aqueous medium comprises membrane dialysis, ultrafiltration, size exclusion chromatography or tangential flow filtration.

3. (canceled)

4. The method of claim 1, wherein said hydrophobic polymer block precursor monomers are cyclic and said polymerization reactions are ring-opening polymerization (ROP) reactions, preferably wherein said hydrophobic polymer block precursor monomers are amino acid N-carboxyanhydrides.

5. (canceled)

6. The method of claim 1, wherein said hydrophobic polymer block comprises a polypeptide.

7. The method of claim 6, wherein said polypeptide comprises amino acid residues selected from the group consisting of methionine, histidine, lysine, glutamic acid, phenylalanine and derivatives thereof, and preferably wherein said polypeptide comprises methionine.

8. (canceled)

9. The method of claim 6, wherein said polypeptide comprises pendant groups having a pKa in the range 4.0 to 7.5.

10. The method of claim 6, wherein said polypeptide comprises chemically reactive pendant groups suitable for further functionalising the self-assembled nanoparticles or microparticles.

11. The method of claim 1, wherein said hydrophilic polymer block comprises a polymer selected from the group consisting of polyesters, polyamides, polyanhydrides, polyurethanes, polyethers, polyimines, polypeptides, polypeptoids, polyureas, polyacetals and polysaccharides.

12. The method of claim 1, wherein said polar aprotic solvent is selected from the group consisting of dimethyl sulfoxide, tetrahydrofuran, dioxane, N,N-dimethyl formamide, N,N-dimethyl acetamide and 1,3-dimethyl-2-imidazolidinone.

13. The method of claim 1, which comprises providing said hydrophilic polymer blocks in situ by polymerizing hydrophilic polymer block precursor monomers.

14. The method of claim 1, wherein steps (i) to (iii) are carried out as a one-pot reaction.

15. The method of claim 1, wherein the reaction steps are terminated when the self-assembled nanoparticles or microparticles constitute from 0.1 to 90% by weight, and preferably from 5 to 70% by weight, of the total weight of the reaction medium.

16. The method of claim 1, wherein the degree of polymerisation of the hydrophobic polymer block is from 5 to 200 and/or wherein the degree of polymerisation of the hydrophilic polymer block is from 5 to 400.

17. The method of claim 1, wherein the self-assembled nanoparticles or microparticles comprise micelles, and optionally wherein the micelles collectively form a gel.

18. The method of any one of claim 1, wherein the self-assembled nanoparticles or microparticles comprise vesicles.

19. A hydrogel comprising a plurality of micellar nanoparticles or microparticles, wherein said nanoparticles or microparticles each comprise amphiphilic copolymers each comprising a hydrophilic polymer block and a hydrophobic polymer block that comprises a polypeptide or polypeptoid.

20. A vesicle that comprises an amphiphilic block copolymer comprising a hydrophilic block and a hydrophobic block that comprises a polypeptide or polypeptoid, wherein said vesicle is suitable for administration to a subject and said hydrophobic block is capable of undergoing a chemical transformation, leading to degradation of said vesicle in vivo, in response to a change in in vivo conditions.

21. The vesicle of claim 20, wherein said change in in vivo conditions is a change in pH and said hydrophobic block comprises pendant groups having a pKa in the range 4.0 to 7.5, optionally wherein said pendant groups comprise imidazolyl groups and preferably wherein at least some of said imidazolyl groups are provided by histidine residues in said hydrophobic block.

22. (canceled)

23. The vesicle of claim 20, wherein said change in in vivo conditions is an increase in the concentration of reactive oxygen species (ROS), optionally wherein said hydrophobic block comprises methionine residues.

24. (canceled)

25. The vesicle of claim 20, wherein a drug is encapsulated within said vesicle or attached to the surface of said vesicle.
